(19) United States
(12) Patent Application Publication
(10) Pub. No.: US 2024/0416032 A1
(43) Pub. Date: Dec. 19, 2024

(54) USER-WEARABLE INFUSION PUMP SYSTEM INCLUDING PUMP HOLDER

(71) Applicant: Tandem Diabetes Care, Inc., San Diego, CA (US)

(72) Inventor: Neel Shah, San Diego, CA (US)

(21) Appl. No.: 18/441,735

(22) Filed: Feb. 14, 2024

Related U.S. Application Data
(60) Provisional application No. 63/445,962, filed on Feb. 15, 2023.

(51) Int. Cl. A61M 5/142

(52) U.S. Cl. CPC A61M 5/14244; A61M 2207/10

(57) ABSTRACT

Embodiments of the present disclosure relate to an infusion pump system including a holder or carrier for a user-wearable infusion pump that can be worn on or near the patient's body. Embodiments depicted and described herein provide a more comfortable yet secure pump holder by providing the holder with a combination of flexible and rigid materials.

### PRIORITY CLAIM

The present application claims the benefit of U.S. Provisional Patent Application No. 63/445,962, which is hereby incorporated herein by reference in its entirety.

### TECHNICAL FIELD

The present disclosure relates to medical pumps for delivering medicament to a patient, and more specifically, to a user-wearable infusion pump that can be worn on the body of a user.

### BACKGROUND

There are a wide variety of medical treatments that include the administration of a therapeutic fluid in precise, known amounts at predetermined intervals. Devices and methods that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

### SUMMARY

Embodiments of the present disclosure relate to an infusion pump system including a holder or carrier for a user-wearable infusion pump that can be worn on or near the patient's body. Embodiments depicted and described herein provide a more comfortable yet secure pump holder by providing the holder with a combination of flexible and rigid materials.

In an embodiment, a user-wearable infusion pump system includes a user-wearable infusion pump configured to contain a medicament and a pump holder configured to releasably hold the user-wearable infusion pump and including an adhesive patch configured to retain the pump holder on a body of a user. The pump holder can include a frame base comprising a flexible material and having an outer perimeter having a shape generally matching a shape of a perimeter of the user-wearable infusion pump and a plurality of pump retention features comprising a rigid material.

In an embodiment, a pump holder for a user-wearable infusion pump can include an adhesive patch configured to retain the pump holder on a body of a user, a frame base comprising a flexible material and having an outer perimeter having a shape generally matching a shape of a perimeter of the user-wearable infusion pump and a plurality of pump retention features comprising a rigid material.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1D depict an embodiment of a pump system according to the disclosure.

FIGS. 2A-2C depict the pump system of FIGS. 1A-1D.

FIGS. 3A-3B depict a holder for an infusion pump according to an embodiment of the disclosure.

FIG. 4 depicts the holder of FIGS. 3A-3B carrying an infusion pump.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

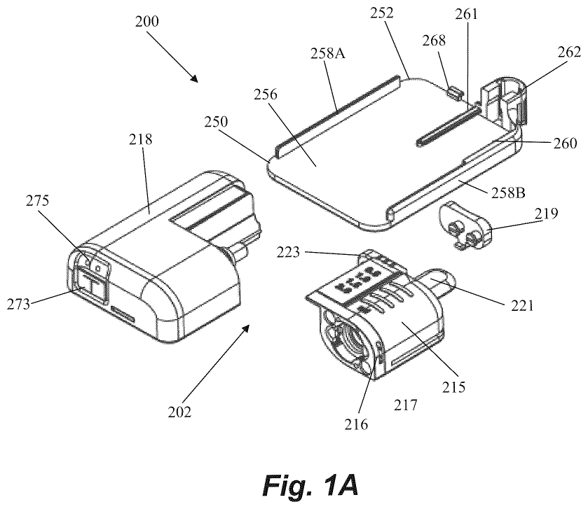

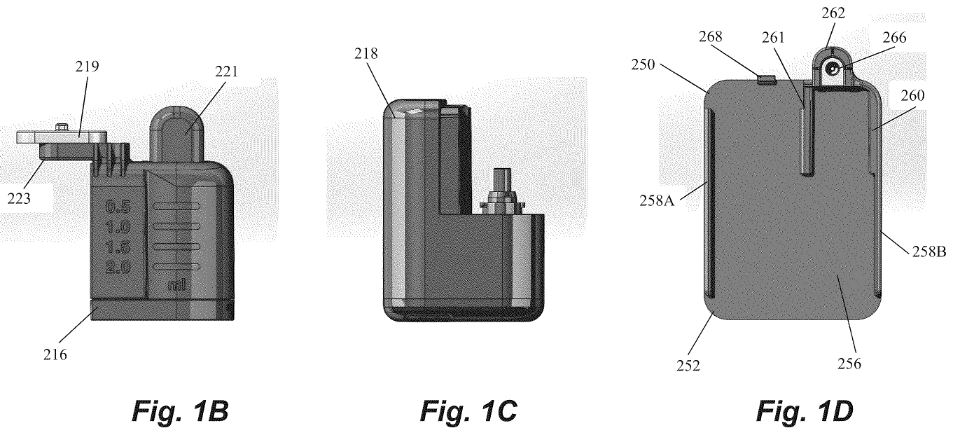

Referring now to FIGS. 1A-1D, an infusion pump system 200 including a user-wearable infusion pump 202 and a pump holder 250 is depicted. Pump can include a cartridge 216 having a reservoir 215 configured to contain a medicament and a drive unit 218. Cartridge can include a cannula interface 221 having an outlet fluidly connected to the interior of the reservoir 215 and configured to interface with a cannula extending below the pump to deliver medicament from the reservoir 215 to the user. A clip or switch 219 can be disposed on cartridge 216 and slide along a switch mount 223 on cartridge to releasably secure the pump 202 to pump holder 250. In some embodiments, cartridge 216 is configured to be refilled while in other embodiments the cartridge 216 is disposable and discarded after a single use to be replaced with a new cartridge.

In embodiments, pump 202 can interface with a cannula that extends through the pump holder 250 to deliver medicament to an infusion site on the user's body directly beneath the pump holder 250. In other embodiments, pump 202 can interface with infusion tubing to deliver medicament from the pump through the tubing to an infusion site displaced from the pump holder 250. In some embodiments, the pump 202 can be configured to interchangeably be used with a cannula directly beneath the pump holder 250 or an infusion site displaced from the holder 250 via infusion tubing. In some of those embodiments, drive unit 218 can be configured to attach to alternative cartridge types, with a first cartridge type including infusion tubing and a second cartridge type configured to deliver through a cannula extending directly beneath the pump 202. In various embodiments, pump 202 can be disconnected from a user's body and reattached using the same infusion site to enable the user to remove the pump for a limited period of time for certain activities, such as, e.g., charging the battery, taking a shower, playing sports, etc. without having to insert a cannula at a new infusion site. Further details regarding such a pump system can be found in U.S. Provisional Patent Application No. 63/397,234, which is incorporated by reference herein in its entirety.

In embodiments, pump includes a processor that controls the operations of the pump and may communicate in either one-way or two-way modes to, e.g., receive operational commands and/or other signals, including data, from a separate device and/or, e.g., to send signals, including data to a separate device. Pump can include on or more buttons configured to cause the processor to initiate one or more functions. In the depicted embodiment, pump 202 includes only a single button 273, although more than one button may be present on pump 202. Button 273 can be configured to, for example, initiate delivery of medicament (e.g., a “quick bolus”). Any single button such as button 273 can be utilized to execute a plurality of functions or operations. For example, a single press of button may initiate one function, holding the button down for a predetermined period of time may initiate another function, etc. Because the depicted pump 202 optionally does not itself include a display or user interface, information and feedback regarding medicament delivery or dosing or other functions initiated with button 273 can be communicated to and displayed on a remote control device or other device having a display and/or other type of user interface.

In embodiments, pump 202 includes a light source, such as a light emitting diode (LED) 275. Light source 275 can be configured to provide user feedback regarding user input and/or the performance of a desired function. For example, in one embodiment, light source 275 can illuminate or blink one or more times to indicate that the one or more buttons 273 have been activated and/or that a desired function has been initiated. In one embodiment, pump 202 can additionally and/or alternatively vibrate and/or provide audible notifications to indicate that the one or more buttons 273 have been activated and/or that a desired function has been initiated or, e.g., to provide user feedback regarding user input and/or the performance of the desired function. Illumination of light source 275 and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In the depicted embodiment, the pump 202 includes two indicator lights 275.

As noted above, infusion pump system 200 can further include a pump holder 250 that can be used to releasably contain a user-wearable infusion pump 202. Holder 250 can include a frame 252 disposed on an adhesive patch (not pictured in this embodiment) configured to attach the pump holder 200 directly to the body of a user. Frame 252 can include a base 256 on which the pump rests and one or more perimeter walls 258A, 258B that surround one or more portions of the perimeter of a pump disposed on base 256. Any of the perimeter walls 258 can include engagement features that aid in retaining pump on holder 250. For example, in the depicted embodiment perimeter wall 258B includes an inwardly extending locking projection 260. When the pump 202 is disposed on the holder 250, a corresponding projection 217 on the cartridge 216 nests beneath the locking projection 260 to aid in retaining the pump 202 on the holder 250. Pump holder 250 can further include a guide rail 261 configured to interface with a corresponding recess in pump 202 to aid in proper alignment of pump 202 on holder 250 and to further aid in retention of pump. Pump holder 250 can also be provided with a locking projection 268 that interfaces with switch 219 on pump to releasably lock the pump 202 on the holder 250. Pump holder 250 can further include a cannula/septum module 262. Cannula/septum module can be sized to receive the cannula interface 221 of the cartridge 216 and to fluidly connect the outlet of the cartridge 216 with a cannula extending through cannula/septum module 262 beneath the pump holder 200 and into the user's skin. In practice, a needle can be inserted through a cannula aperture 266 to insert the cannula into the skin and then removed prior to inserting the pump 202 onto the pump holder 250, leaving the cannula in place.

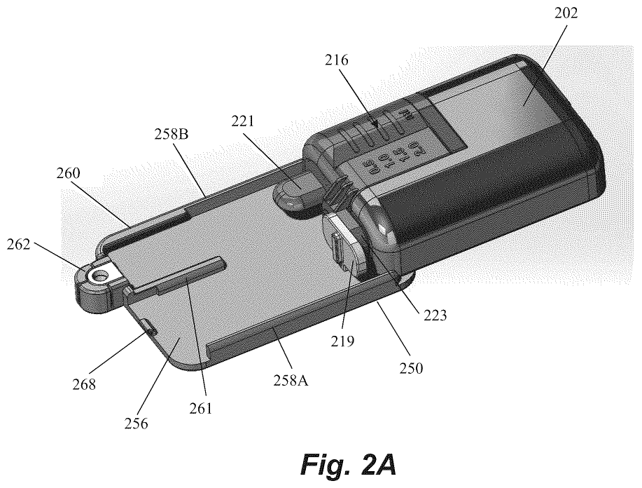

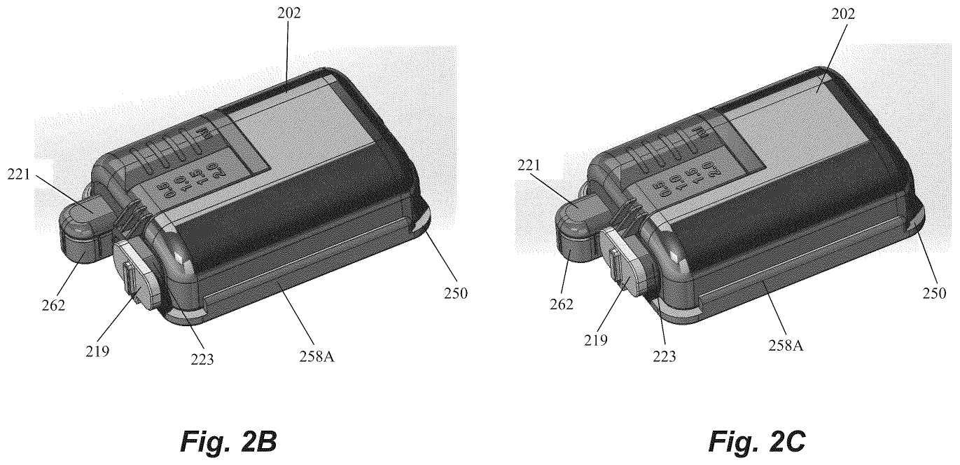

Referring now to FIGS. 2A-2C, pump 202 can be inserted onto pump holder 250 by first positioning the perimeter of pump between perimeter walls 258A, 258B as depicted in FIG. 2A. The pump 202 can then be slid along the frame base 256 such that the guide rail 261 on the holder 250 interfaces with the corresponding feature on the bottom side of the pump and the projection 217 on pump slides beneath the locking projection 260 of retention wall 258B until the cannula interface 221 on pump is mated with the septum/cannula module 262 of the holder, as shown in FIG. 2B. To releasably lock the pump 202 on the holder 250, the switch 219 on cartridge 216 is then slid laterally along the switch mount 223 (towards the cannula interface 221) from an unlocked position (FIG. 2B) to a locked position (FIG. 2C). The switch 219 interface with the locking projection 268 on the holder 250 to releasably lock the pump on the holder 250. In some embodiments, switch 219 can be spring-loaded and configured to automatically transition to the locked position when the pump 202 is seated on the holder 250.

In the embodiment described above, holder 250 generally comprises a unitary construct formed of a rigid material such as plastic. However, because the holder is worn directly on the body, a rigid construction may be uncomfortable for the user in certain body locations. Yet, if the entire holder is formed of a flexible material the pump may not be adequately retained on the tray or on the body of the user. Embodiments described herein therefore provide pump holders 350 that comprise a combination of rigid and flexible materials in order to provide a more comfortable holder for the user to wear while retaining critical pump retention features.

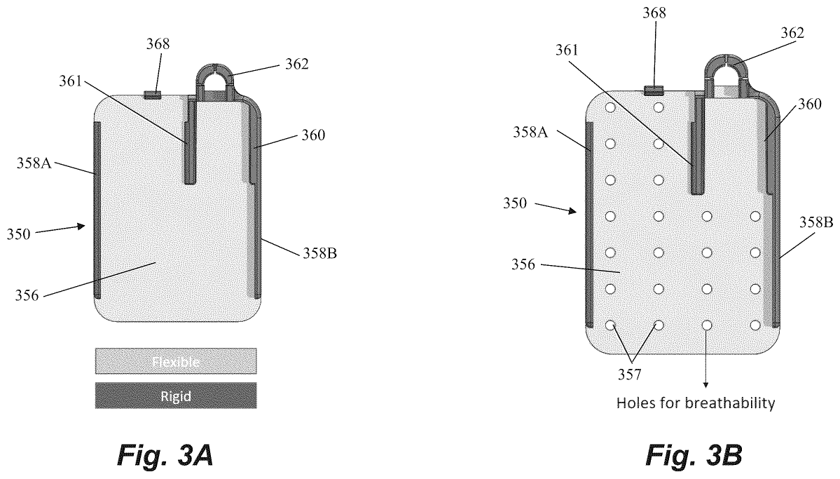

Referring now to FIGS. 3A-3B, pump holders 350 including a combination of rigid and flexible materials are depicted. Rigid materials can be employed for critical pump retention and alignment features, such as, for example, one or more of retention wall 358A, retention wall 358B (including locking projection 360), locking projection 368 and guide rail 361. These features can be provided as rigid materials in order to provide them with the required strength for interfacing with the pump. Cannula module 262 can also be comprised of a rigid material. Such rigid materials can include, for example, polycarbonate, polypropylene, acrylonitrile-butadiene-styrene and other similar materials. Frame base 356 can be comprised of a flexible, compliant material. Such flexible materials can include, for example, silicone, thermoplastic elastomer and thermoplastic polyurethane. As this is the feature directly in contact with the user, use of such a material as the base 356 provides elasticity at the infusion site for a more comfortable experience for the user. Providing the components noted above as rigid materials enables the pump to still be firmly retained on the holder 350. In one embodiment, rigid features can be overmolded onto flexible base 356. FIG. 3B depicts an embodiment that includes a plurality of apertures 357 through flexible frame base 356 that can provide enhanced breathability to the holder 350 for further enhancement to user comfort.

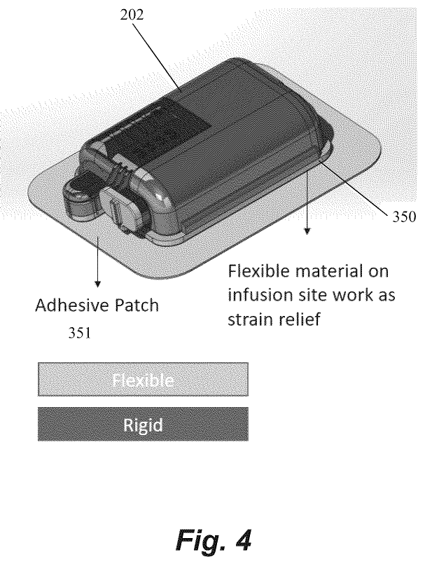

As shown in FIG. 4, the flexible base 356 can be bonded to an adhesive patch 351 that affixes the holder 350 to the user's body. The elasticity provided by a base formed of flexible material bonded with adhesive patch, can further provide strain relief at the connection point relative to a rigid base to help with the infusion site survival rate.

Although one specific embodiment of a pump holder having a combination of rigid and flexible component is described herein, it should be understood that the general concept can be applied to any other user-wearable infusion pump holder. For example, any of the pump holders disclosed in U.S. Pat. No. 10,279,106, U.S. Patent Publication No. 2023/0173170 and U.S. patent application Ser. Nos. 18/448,584 and 18/407,663, 18/each of which is incorporated by reference herein in its entirety, could be configured to employ a combination or rigid and flexible materials as taught herein.

Although the embodiments herein have been specifically described with respect to a user-wearable infusion pump, the inventions disclosed herein could be employed with any other type of medical device capable of being worn on or near the body. Embodiments could further include non-medical applications, including smartphones. For example, a smartphone or other device could be placed into carriers such as those described herein to be interchangeably attached to various accessories.

In an embodiment, a user-wearable infusion pump system includes a user-wearable infusion pump configured to contain a medicament and a pump holder configured to releasably hold the user-wearable infusion pump and including an adhesive patch configured to retain the pump holder on a body of a user. The pump holder can include a frame base comprising a flexible material and having an outer perimeter having a shape generally matching a shape of a perimeter of the user-wearable infusion pump and a plurality of pump retention features comprising a rigid material.

In some embodiments, the pump retention features comprising a rigid material include one or more retention walls extending upwardly from the outer perimeter of the frame base.

In some embodiments, the pump retention features comprising a rigid material include one or more guide rails extending upwardly from the frame base interior of the outer perimeter of the frame base.

In some embodiments, the one or more guide rails are configured to interface with a corresponding recess on the user-wearable infusion pump to aid in proper alignment of the user-wearable infusion pump on the pump holder.

In some embodiments, the pump retention features comprising a rigid material include a locking projection extending upwardly from the outer perimeter of the frame base.

In some embodiments, the locking projection is configured to interface with a switch on the user-wearable infusion pump that can slide between locked and unlocked positions to releasably lock the user-wearable infusion pump on the pump holder.

In some embodiments, the pump retention features comprising a rigid material include a cannula module configured to receive a cannula inserted there through.

In some embodiments, the cannula module is configured to interface with a cannula interface of the user-wearable infusion pump to fluidly connect an outlet of the user-wearable infusion pump with the cannula.

In some embodiments, the rigid material is selected from the set consisting of polycarbonate, polypropylene and acrylonitrile butadiene styrene.

In some embodiments, the flexible material is selected from the set consisting of silicone, thermoplastic elastomer and thermoplastic polyurethane.

In an embodiment, a pump holder for a user-wearable infusion pump can include an adhesive patch configured to retain the pump holder on a body of a user, a frame base comprising a flexible material and having an outer perimeter having a shape generally matching a shape of a perimeter of the user-wearable infusion pump and a plurality of pump retention features comprising a rigid material.

In some embodiments, the pump retention features comprising a rigid material include one or more retention walls extending upwardly from the outer perimeter of the frame base.

In some embodiments, the pump retention features comprising a rigid material include one or more guide rails extending upwardly from the frame base interior of the outer perimeter of the frame base and the one or more guide rails can be configured to interface with a corresponding recess on a user-wearable infusion pump to aid in proper alignment of the user-wearable infusion pump on the pump holder.

In some embodiments, the pump retention features comprising a rigid material include a locking projection extending upwardly from the outer perimeter of the frame base and the locking projection configured to interface with a switch on a user-wearable infusion pump that can slide between locked and unlocked positions to releasably lock the user-wearable infusion pump on the pump holder.

In some embodiments, the pump retention features comprising a rigid material include a cannula module configured to receive a cannula inserted therethrough and the cannula module can be configured to interface with a cannula interface of a user-wearable infusion pump to fluidly connect an outlet of the user-wearable infusion pump with the cannula.

In some embodiments, the rigid material is selected from the set consisting of polycarbonate, polypropylene and acrylonitrile butadiene styrene.

In some embodiments, the flexible material is selected from the set consisting of silicone, thermoplastic elastomer and thermoplastic polyurethane.

In embodiments, a method of manufacturing a pump holder for a user-wearable infusion pump can include providing a frame base comprising a first material and having an outer perimeter having a shape generally matching a shape of a perimeter of a user-wearable infusion pump. The method can include overmolding a plurality of pump retention features comprising a second material on to the frame base, wherein the second material is more rigid than the first material.

In some embodiments, the second material is selected from the set consisting of polycarbonate, polypropylene and acrylonitrile butadiene styrene.

In some embodiments, the first material is selected from the set consisting of silicone, thermoplastic elastomer and thermoplastic polyurethane.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patents applications Ser. Nos. 17/368,968; 17/896,492; 18/011,060; 18/071,814; 18/071,835; 18/075,029; 18/090,788 18/115,316; 18/139,391; 18/398,543; 18/407,663; 18/407,915; 18/475,916; 18/478,552 and 18/568,950.

For purposes of interpreting the claims, it is expressly intended that the provisions of 35 U.S.C. § 112(f) are not to be invoked unless the specific terms “means for” or “step for” are recited in a claim.

1. A user-wearable infusion pump system, comprising:
a user-wearable infusion pump configured to contain a medicament;
a pump holder configured to releasably hold the user-wearable infusion pump and including an adhesive patch configured to retain the pump holder on a body of a user, the pump holder comprising:
a frame base comprising a flexible material and having an outer perimeter having a shape generally matching a shape of a perimeter of the user-wearable infusion pump; and
a plurality of pump retention features comprising a rigid material.

2. The user-wearable infusion pump system of claim 1, wherein the pump retention features comprising a rigid material include one or more retention walls extending upwardly from the outer perimeter of the frame base.

3. The user-wearable infusion pump system of claim 1, wherein the pump retention features comprising a rigid material include one or more guide rails extending upwardly from the frame base interior of the outer perimeter of the frame base.

4. The user-wearable infusion pump system of claim 3, wherein the one or more guide rails are configured to interface with a corresponding recess on the user-wearable infusion pump to aid in proper alignment of the user-wearable infusion pump on the pump holder.

5. The user-wearable infusion pump system of claim 1, wherein the pump retention features comprising a rigid material include a locking projection extending upwardly from the outer perimeter of the frame base.

6. The user-wearable infusion pump system of claim 5, wherein the locking projection is configured to interface with a switch on the user-wearable infusion pump that can slide between locked and unlocked positions to releasably lock the user-wearable infusion pump on the pump holder.

7. The user-wearable infusion pump of claim 1, wherein the pump retention features comprising a rigid material include a cannula module configured to receive a cannula inserted therethrough.

8. The user-wearable infusion pump of claim 7, wherein the cannula module is configured to interface with a cannula interface of the user-wearable infusion pump to fluidly connect an outlet of the user-wearable infusion pump with the cannula.

9. The user-wearable infusion pump of claim 1, wherein the rigid material is selected from the set consisting of polycarbonate, polypropylene and acrylonitrile butadiene styrene.

10. The user-wearable infusion pump of claim 1, wherein the flexible material is selected from the set consisting of silicone, thermoplastic elastomer and thermoplastic polyurethane.

11. A pump holder for a user-wearable infusion pump, comprising:
an adhesive patch configured to retain the pump holder on a body of a user;
a frame base comprising a flexible material and having an outer perimeter having a shape generally matching a shape of a perimeter of a user-wearable infusion pump; and
a plurality of pump retention features comprising a rigid material.

12. The pump holder of claim 11, wherein the pump retention features comprising a rigid material include one or more retention walls extending upwardly from the outer perimeter of the frame base.

13. The pump holder of claim 11, wherein the pump retention features comprising a rigid material include one or more guide rails extending upwardly from the frame base interior of the outer perimeter of the frame base, the one or more guide rails configured to interface with a corresponding recess on a user-wearable infusion pump to aid in proper alignment of the user-wearable infusion pump on the pump holder.

14. The pump holder of claim 11, wherein the pump retention features comprising a rigid material include a locking projection extending upwardly from the outer perimeter of the frame base, the locking projection configured to interface with a switch on a user-wearable infusion pump that can slide between locked and unlocked positions to releasably lock the user-wearable infusion pump on the pump holder.

15. The pump holder of claim 11, wherein the pump retention features comprising a rigid material include a cannula module configured to receive a cannula inserted therethrough, the cannula module configured to interface with a cannula interface of a user-wearable infusion pump to fluidly connect an outlet of the user-wearable infusion pump with the cannula.

16. The pump holder of claim 11, wherein the rigid material is selected from the set consisting of polycarbonate, polypropylene and acrylonitrile butadiene styrene.

17. The pump holder of claim 11, wherein the flexible material is selected from the set consisting of silicone, thermoplastic elastomer and thermoplastic polyurethane.

18. A method of manufacturing a pump holder for a user-wearable infusion pump, comprising:
providing a frame base comprising a first material and having an outer perimeter having a shape generally matching a shape of a perimeter of a user-wearable infusion pump; and
overmolding a plurality of pump retention features comprising a second material on to the frame base, wherein the second material is more rigid than the first material.

19. The method of claim 18, wherein the second material is selected from the set consisting of polycarbonate, polypropylene and acrylonitrile butadiene styrene.

20. The method of claim 18, wherein the first material is selected from the set consisting of silicone, thermoplastic elastomer and thermoplastic polyurethane.
